(19) United States
(12) Patent Application Publication
(10) Pub. No.: US 2005/0281891 A1
(43) Pub. Date: Dec. 22, 2005

(54) Testosterone stimulant

(75) Inventors: Suk Jung Oh, Gwangju (KR); Kwang Yoon Kim, Gwangju (KR)

Correspondence Address: THORPE NORTH & WESTERN, LLP., 8180 SOUTH 700 EAST, SUITE 200, P.O. BOX 1219, SANDY, UT, 84070, US

(73) Assignee: Ecobio, Inc.

(21) Appl. No.: 11/145,545

(22) Filed: Jun. 2, 2005

Related U.S. Application Data
(60) Provisional application No. 60/576,987, filed on Jun. 3, 2004.

(51) Int. Cl. A61K 31/722; A61K 31/375; A61K 33/32; A61K 35/80

(52) U.S. Cl. 424/641; 514/55; 424/195.17; 514/474

(57) ABSTRACT

Methods of increasing blood testosterone levels of a subject can comprise administering an iron chitosan chelate to the subject. The chitosan component can have a weight average molecular weight from about 0.5 kDa to about 50 kDa. The iron chitosan chelate can be co-administered with at least one ingredient selected from the group consisting of ascorbic acid, kelp, iron, and zinc. Additionally, iron chitosan chelates can be formed by hydrolyzing source chitosan to form a hydrolyzed chitosan mixture, wherein the chitosan mixture includes a desired portion; separating the desired portion from the chitosan mixture; and chelating the desired portion with iron.

The present non-provisional application claims the benefit of U.S. Provisional Application No. 60/60/576,987 filed Jun. 3, 2004, which is incorporated herein by reference.

### FIELD OF THE INVENTION

The present invention relates generally to steroid hormone stimulation. More particularly, the present invention relates to the use of iron chitosan chelates to stimulate natural testosterone production.

### BACKGROUND OF THE INVENTION

Most steroid hormones are derived from and contain the same cyclopentanophenanthrene ring structure as cholesterol. Common steroid hormones include progesterone, aldosterone, testosterone, estradiol, and cortisol. The steroid hormones typically exert their physiological action by passing through a plasma membrane of a cell and then binding to intracellular receptors.

Testosterone and estradiol are important sex steroid hormones, and are mainly produced by the testes and the ovaries, respectively. These sex hormones are produced under tight biosynthetic control, with short and long feedback loops that regulate the secretion of leutinizing hormone (LH) and follicle stimulating hormone (FSH) from the pituitary gland by controlling the secretion of gonadotropin releasing hormone (GnRH) from the hypothalamus. Low levels of sex steroid hormones circulating in the blood can increase GnRH synthesis, which in turn leads to elevated FSH and LH levels. In male humans, LH binds to associated biosynthetic pathways can have an affect on the amount of hormone produced and/or the sex steroid hormone level in the blood.

Testosterone is responsible for normal growth and development of male sex and reproductive organs, including the seminal vesicles, scrotum, prostate, testicles, and penis. Additionally, testosterone can facilitate the development of male sex characteristics such as laryngeal enlargement, vocal chord thickening, hair patterns, fat distribution, bone mass, and musculature. Normal testosterone levels maintain secondary sexual characteristics such as normal energy levels, mood, fertility, and sexual desire.

Testosterone production declines naturally with age, which can result in testosterone deficiency (TD). Additionally, disease or damage to the hypothalamus, pituitary gland, or testicles can interfere with the biosynthesis controls and can result in inhibited hormone secretion and lowered testosterone production. Testosterone deficiency can also be caused by chemotherapy, radiation, testicular trauma, head trauma affecting the hypothalamus, and infections such as meningitis, syphilis, and mumps. Signs and symptoms of testosterone deficiency can be exhibited by men experiencing diminished libido, erectile dysfunction, muscle weakness, loss of body hair, depression, and other mood disorders.

Treatment of testosterone deficiency usually involves hormone replacement therapy. The treatment for adults is usually aimed at maintaining the secondary sex characteristics, such as by transdermal, mucoadhesive, and intramuscular modes of delivery. Orally delivered testosterone has been associated with liver toxicity and liver tumors, and thus is prescribed sparingly. Hormone replacement therapy can cause some men to experience fluid retention, acne, and temporary abnormal breast development also known as gynecosmastia.

In accordance with the treatment or prevention of testosterone deficiency, therapeutics that can naturally increase the amount of steroid hormone in the blood and improve secondary sex characteristics without delivering replacement hormones continue to be sought through research and development.

### SUMMARY OF THE INVENTION

It has been recognized that it would be advantageous to provide methods of stimulating testosterone production in subjects needing increased levels of testosterone. As such, the present invention provides methods of increasing testosterone levels in a subject that can comprise identifying a subject having lower than average blood testosterone levels and administering a therapeutically effective amount of an iron chitosan chelate to the subject such that blood testosterone level of the subject is increased.

In one embodiment, methods of increasing testosterone levels in a subject can comprise identifying a subject having lower than average blood testosterone levels; administering a therapeutically effective amount of chitosan to the subject; and co-administering at least one ingredient selected from the group consisting of ascorbic acid, iron, zinc, and kelp to the subject such that the blood testosterone level of the subject is increased.

In another embodiment, a composition for enhancing testosterone stimulation can comprise an iron chitosan chelate and at least one ingredient selected from the group consisting of ascorbic acid, kelp, and a zinc source. The chitosan can have a weight average molecular weight from about 0.5 kDa to about 50 kDa.

Also, in accordance with the present invention, a method of preparing an iron chitosan chelate can comprise steps of hydrolyzing source chitosan to form a hydrolyzed chitosan mixture, wherein the chitosan mixture includes a desired portion and a remainder portion; separating the desired portion from the remainder portion; and chelating the desired portion with iron.

In yet another embodiment, a method of preparing a composition for enhancing testosterone stimulation can comprise steps of preparing an iron chitosan chelate as recited above, and combining the iron chitosan chelate with at least one ingredient selected from the group consisting of ascorbic acid, kelp, and zinc.

Additional features and advantages of the invention will be apparent from the detailed description that follows, which illustrates, by way of example, features of the invention.

### DETAILED DESCRIPTION OF THE INVENTION

Before the present invention is disclosed and described, it is to be understood that this invention is not limited to the particular process steps and materials disclosed herein because such process steps and materials may vary somewhat. It is also to be understood that the terminology used herein is used for the purpose of describing particular embodiments only. The terms are not intended to be limiting, as the scope of the present invention is to be limited only by the appended claims and equivalents thereof.

It must be noted that, as used in this specification and the appended claims, the singular forms “a,” “an,” and “the” include plural referents unless the context clearly dictates otherwise.

As used herein, “chitosan” refers to a linear polymer having Formula I, as follows: 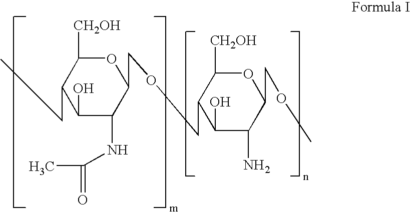
where the deacetylated n groups comprise greater than 70% and the acetyl m groups comprise less than 30% of the polymers. Additionally, it should be understood that chitosan is a natural product derived by deacetylation of chitin, a polysaccharide often found in the exoskeleton of shellfish such as shrimp or crabs. Chitin is a naturally occurring substance that may be regarded as a derivative of cellulose having predominantly linear chains. Typically, chitosan is said to exist when chitin has been deacetylated by more than 70%.

As used herein, “chelate,” refer to chemical compounds in the form of a heterocyclic ring, wherein the ring includes at least one organic ligand and a metal ion as a closing member. The ligand, in accordance with embodiments of the present invention, can be chitosan, and the metal can be ferric or ferrous iron.

When using the plural term “chelates,” this phraseology is not intended to necessarily require the presence of two distinct types of chelates, but can include only a single species of a chelate. Alternatively, the term “chelates” can also include multiple types of chelate species. Such distinctions can be easily ascertained and depend on the context of use.

As used herein, “ultrafiltration” refers to the filtration of a mixture containing polymers through a semi-permeable medium that allows only the passage of small polymers and small molecules. Accordingly, the selection of the semi-permeable medium can allow for selecting an average molecular weight cut-off, which prevents molecules and polymers above this average molecular weight cut-off from passing through the medium. Thus, by ultrafiltration, polymers and molecules smaller than the cut-off can be selected for use as they are able to pass through the medium for collection.

The terms “active agent,” “bioactive agent,” and “pharmaceutically active agent,” can also be used interchangeably to refer to an agent or substance that has measurable specified or selected physiologic activity when administered to a subject in an effective amount. These terms of art are well-known in the pharmaceutical, nutraceutical, and medicinal arts.

As used herein, the terms “administration,” and “administering” can refer to the manner in which a dosage form, formulation, or composition is introduced into the body of a subject. Administration can be accomplished by various art-known routes such as oral, parenteral, transdermal, inhalation, implantation, etc. Oral administration can be achieved by swallowing, chewing, and/or sucking of an oral dosage form comprising an active agent. Parenteral administration can be achieved by injecting an iron chitosan chelate-containing composition intravenously, intra-arterially, intramuscularly, intrathecally, or subcutaneously, etc. Transdermal administration can be accomplished by applying, affixing, pasting, rolling, attaching, pouring, pressing, rubbing, etc., of a transdermal preparation onto a skin surface. These and additional methods of administration are well-known in the art.

The terms “effective amount” and “sufficient amount” can be used interchangeably and refer to an amount of an ingredient which, when included in a composition, is sufficient to achieve an intended compositional or physiological effect. Thus, a “therapeutically effective amount” refers to a non-toxic, but sufficient amount, of an active agent to achieve therapeutic results in treating a condition for which the active agent can be effective. Various biological factors can affect the ability of a substance to perform its intended task. Therefore, an “effective amount”- or a “therapeutically effective amount” can be dependent on such biological factors. Further, while the achievement of therapeutic effects can be measured by a physician or other qualified medical personnel using evaluations known in the art, it is recognized that individual responses to treatments can make the achievement of therapeutic effects variable. Accordingly, the determination of an effective amount is well within the ordinary skill in the art of pharmaceutical, nutraceutical, medicinal arts, and health sciences.

As used herein, the terms “carrier” or “pharmaceutically acceptable carrier” refers to a carrier vehicle with which a bioactive agent may be combined to achieve a specific dosage form. As a general principle, carriers do not react with the bioactive agent in a manner which substantially degrades or otherwise adversely affects the bioactive agent. However, a “carrier” can interact with the bioactive agent so long as the beneficial activity of the agent is not significantly diminished or defeated. Other “inactive” ingredients may also be used in creating chelate, chitosan, and/or nutritionally relevant multivalent metal source containing formulations having specifically desired properties or dosage forms, and will be readily recognized by those of ordinary skill in the art.

As used herein, the term “subject” refers to an animal, which is usually a mammal, that can benefit from the administration of compositions in accordance with embodiments of the present invention. Most often, the subject can be a human.

The term “secretion” refers to the physiological processes of substance generation and/or separation from either the cells or bodily fluids of a subject. Thus, the secretion of testosterone refers to the physiological process for releasing testosterone from a cell into an extra-cellular space, which includes starting with the intra-cellular machinery for building and releasing LH, FSH, and GnRH, and resulting in the release of testosterone into systemic circulation.

The term “about” when referring to a numerical value or range is intended to encompass the values resulting from experimental error that can occur when taking measurements.

Concentrations, amounts, molecular sizes, and other numerical data may be expressed or presented herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 wt % to about 5 wt %” should be interpreted to include not only the explicitly recited concentration of about 0.1 wt % to about 5 wt %, but also include individual concentrations and the sub-ranges within the indicated range. Thus, included in this numerical range are individual concentrations such as 1 wt %, 2 wt %, 3 wt %, and 4 wt %, and sub-ranges such as from 0.1% to 1.5 wt %, 1% to 3 wt %, from 2% to 4 wt %, from 3% to 5 wt %, etc. This same principle applies to ranges reciting only one numerical value. For example, a range recited as “less than about 5 wt %” should be interpreted to include all values and sub-ranges between 0% and 5 wt %. Furthermore, such an interpretation should apply regardless of the breadth of the range or the characteristics being described.

In accordance with the present invention, methods and compositions are disclosed for increasing testosterone levels. In one embodiment, a method of increasing testosterone levels can comprise identifying a subject having lower than average blood testosterone levels, and administering a therapeutically effective amount of an iron chitosan chelate to the subject such that blood testosterone level of the subject is increased. The method can additionally include co-administering at least one ingredient selected from the group consisting of ascorbic acid, kelp, and a zinc source. Thus, the iron chitosan chelate can be administered in a common dosage form with any or all of the additional ingredients.

In another embodiment of the present invention, a method of increasing testosterone levels in a subject can comprise identifying a subject having lower than average blood testosterone levels, and administering a therapeutically effective amount of chitosan to the subject. Additionally, the method can include co-administering a therapeutically effective amount of at least one ingredient selected from the group consisting of ascorbic acid, iron, zinc, and kelp to a subject such that blood testosterone level of the subject is increased. In one aspect, the ingredient can be ascorbic acid administered in a therapeutically effective amount. In another aspect, the ingredient is iron chelated to the chitosan compound to form an iron chitosan chelate.

Regarding the chitosan, the chitosan material can be chelated to iron in one embodiment, and/or can be free in another embodiment. Also, the chitosan can have a weight average molecular weight from about 0.5 kDa to about 50 kDa. Alternatively, the chitosan can have a weight average molecular weight from about 0.5 kDa to about 20 kDa. Chitosan has been shown to have various physiological effects when administered to a subject, such as to decrease cholesterol and other lipids, and to increase urinary calcium excretion.

Regarding the subject, typically, the compositions and methods of the present invention are primarily for humans, particularly male humans, though other animals that have a need for testosterone can benefit from the present invention as well. Specific dosage regimens that are sufficient to achieve and/or maintain increased testosterone levels in the subject over a defined period of time may be determined by those of ordinary skill in the art. The daily dosages can depend, to some degree, on which of the ingredients are included in the composition to be delivered. For example, if the composition includes only an iron chitosan chelate and carrier/filler, then more iron chitosan chelate may be desirable to administer compared to a composition that includes other ingredients that contribute to testosterone stimulation. Such amounts to administer on a case by case basis would be easily ascertainable by one skilled in the art after considering the present disclosure

Another embodiment of the present invention provides a composition containing an ingredient selected from the group consisting of an iron chitosan chelate and at least one ingredient selected from the group consisting of ascorbic acid, kelp, and zinc. In such an iron chitosan chelate-containing composition, the chitosan can have a weight average molecular weight from about 0.5 kDa to about 50 kDa. In another aspect, the composition can also include an organic acid selected from the group consisting of lactic acid, acetic acid, citric acid, malic acid, and combinations thereof. Typically, the organic acid is used to prepare the iron chitosan chelate, and remains present in the final composition. Such compositions may be suitably prepared into a number of dosage forms for administration to a subject by combination with additional ingredients, such as carriers, fillers, etc. Examples of suitable dosage forms include liquids, e.g., solutions or suspension, and solids, e.g., granules, freeze-dried powders, and spray dried powders. These dosage forms can be delivered by one or more of many routes, such as oral, parenteral, transdermal, and/or transmucosal delivery.

In another embodiment, a method of preparing an iron chitosan chelate can comprise the steps of hydrolyzing source chitosan to form a hydrolyzed chitosan mixture, wherein the chitosan mixture includes a desired portion and a remainder portion; separating the desired portion from the remainder portion; and chelating the desired portion with iron. Generally, the hydrolyzing step can occur by mixing the chitosan source with chitosanase in the presence of an organic acid. In one aspect, the organic acid can be acetic acid. Alternatively, the hydrolyzing step can occur by mixing the chitosan source with chitosanase in the presence of hydrochloric acid. Additionally, the method can include the step of heating the mixture prior to the separating step to stop hydrolysis of the chitosan. The desired portion of the chitosan mixture can be separated out using ultrafiltration or other techniques known in the art. In one embodiment, the desired portion of the hydrolyzed chitosan can have a weight average molecular weight from about 0.5 kDa to about 50 kDa, and in another embodiment, the desired portion can have a weight average molecular weight from about 0.5 kDa to about 20 kDa. Additionally, desired portion can prepared in solution such that less than about 10% to 15% w/v of unhydrolyzed chitosan remains, and where chitostran strands having a weight average molecular weight above about 100 kDa are discarded.

During the hydrolyzing step, the chitosan chain length can be modified by changing the reaction conditions, where the average molecular weight of chitosan is related to the number of monomer units within the linear chain. For example, increasing the amount of chitosan source can reduce hydrolysis and result in longer polymer chains and higher molecular weight chitosan. However, reducing the amount of chitosan has just the opposite effect because the hydrolysis is able to cleave the polymers into smaller lengths. Alternatively, increasing the amount of chitosanase can result in shorter polymer chains and lower molecular weight chitosan. Further, altering the organic acid can also affect the size of the hydrolyzed chitosan product, e.g., the type and/or the amount of organic acid. Accordingly, one of ordinary skill in the art will realize that modulation of the various reaction parameters can result in the ability to reproduce certain preferred or desired weight average molecular weight ranges of chitosan.

A preferred general manufacturing process for preparing a composition in accordance with embodiments of the present invention is provide below, which is to be emphasized is exemplary only. In preparing compositions in accordance with the present invention, a liquid composition can be prepare, which is optionally dried to form a solid composition. These compositions can become liquid dosage forms and solid dosage forms, respectively, or can be further modified for use within other liquid and solid dosage forms. In the preparatory process, a first step can include a chitosan dissolution step, where from about 0.1% to about 10% (w/v) of chitosan is dissolved. Though a greater amount of chitosan could be dissolved in solution, for practical reasons, this is difficult because of the high viscosity chitosan. Once in solution (or simultaneously added), an acid, such as an organic acid, is added to the chitosan dissolution composition. The amount added can correspond to a chitosan to organic acid weight ratio of about 1:0.2 to about 1:1, depended on organic acid selected for use, e.g., acetic acid can be from about 1:0.2 to about 1:0.4 by weight, and ascorbic acid can be from about 1:0.5 to about 1:0.7 by weight. Thus, in the liquid dissolution, the concentration of organic acids can be from about 0.02% to about 10% (w/v).

After dissolution, chitosanase is added to dissolve chitosan in the organic acid solution for hydrolysis of the chitosan polymer. Optionally, an ultrafiltration step can be carried out to remove undesired portions of the chitosan. Upon addition of iron, the iron chitosan chelate can be formed. For example, an iron salt can be added to the hydrolyzed chitosan solution at a chitosan to ferrous or ferric ion weight ratio from about 1: 0.001 to about 1:0.1. Optionally, additional ingredients can be formulated with the iron chitosan chelate to form compositions. Such additional ingredients can include zinc, vitamin C (ascorbic acid), and/or kelp, for example. Exemplary amounts of each added ingredient can be as follows: chitosan to zinc ion weight ratio from about 1:0.001 to about 1:0.05; chitosan to vitamin C (ascorbic acid) weight ratio from about 1:0.01 to about 1:0.1; and chitosan to kelp weight ratio from about 1:0.005 to about 1:0.1.

After forming the liquid composition in accordance with embodiments of the present invention, a drying step can be carried out to convert the liquid dosage form to a solid dosage form. After spray drying or freeze drying, for example, the weight ratio of chitosan to the other ingredients can be as follows: chitosan to organic acid (excluding ascorbic acid) weight ratio from about 1:0.2 to about 1:1; chitosan to ferric/ferrous iron weight ratio from about 1:0.001 to about 1:0.1; chitosan to zinc weight ratio from about 1:0.001 to about 1:0.05; chitosan to vitamin C weight ratio from about 1:0.01 to about 1:0.1; and/or chitosan to kelp weight ratio from about 1:0.005 to about 1:0.1. These ratios are not to be considered to be limiting, as they are exemplary only. Other weight percentages and concentrations will also be provided herein which may or may not fall within these ranges, and are still considered to be within the teachings of the present invention.

The amount of chitosan present in the compositions of the present invention can be varied based on various factors including the amount and concentration of the various other constituents. In addition, the concentration of chitosan can be dependent on the amount of the composition to be administered. In one aspect, the chitosan can be present in the solid dosage form composition at from about 10 wt % to about 80 wt %. In another aspect, the chitosan is can be present at from about 40 wt % to about 80 wt %. The above weight percentages represent possible amounts that can be present in one type of solid dosage form. As mentioned above, it should be noted that in preparing the composition of the present invention, the chitosan can be added to solution at from 0.1% to 10% (w/v). The concentration of chitosan for daily delivery dosages can depend to some degree on whether other ingredients are included in the composition, as described herein.

In each of the embodiments herein where a composition is prepared in the form of a liquid dosage form or a solid dosage form, the iron chitosan chelate or chitosan (and other optional components) can be carried by a carrier, such as a pharmaceutically acceptable carrier. The carrier can be selected from the group consisting of amino acids or organic acids, hydroscopicity minimizing agents, fillers, flow control agents, lubricants, emulsifiers, oils, disintegrating agents, flow agents, pH control agents, dust control agents, binders, flavoring agents, taste-reducing agents, vitamins, capsule shells, shellacs, waxes, or the like. Specific additives that can be present include calcium carbonate, calcium silicate, calcium magnesium silicate, calcium phosphate, kaolin, sodium hydrogen carbonate, sodium sulfate, barium carbonate, barium sulfate, magnesium sulfate, magnesium carbonate, activated carbon, water, isopropyl alcohol, ethyl alcohol, polyvinyl pyrrolidone, propylene glycol, polyethylene glycol stearyl alcohol, stearic acid, sorbitan monooleate, microcrystalline cellulose, sodium carboxymethyl cellulose, hydroxyethyl cellulose, hydroxypropyl cellulose, hydroxypropyl methylcellulose, sorbitol, mannitol, xylitol, starches, gelatins, lactose, acacia, carbomer, dextrin, guar gum, lactose, liquid glucose, maltodextrin, polymethacrylates, chitin, chitosan (where chitosan is not the active ingredient), polysaccharide, cyclodextrin and combinations thereof.

In one aspect of the present invention, the iron source can be provided as an iron salt. The addition of iron to almost any acid can result in the generation of an iron salt or chelate of that acid. Suitable forms of the iron salts that can be used include ferrous sulfate, ferrous lactate, and combination thereof, to name a few. Accordingly, it is within the scope of this invention for the iron source to be in the form of any iron complex, including all ferrous and ferric complexes. The amount of iron present, once disassociated in solution, can be dependent on the other constituents in the composition as well as the amount of the composition to be administered. Though the concentration for delivery can vary, as described previously, in one embodiment, the iron content can be present in the solid dosage form at from about 0.1 wt % to about 4 wt %. In liquid dosage forms, the iron content of the total composition can be from about 0.05 wt % to about 0.5 wt %, depending on the amount of liquid carrier present. In another embodiment, the iron content can be present in the dosage form at from about 0.1 wt % to about 0.2 wt %. These weight percentages are exemplary, and are based on the dosage form as a whole. With respect to the iron chitosan chelates per se, the iron content of the iron chitosan chelate can be from about 0.1 wt % to about 10 wt %, and in another embodiment, from about 0.5 wt % to about 4 wt %.

In one embodiment, the chitosan component can be present in molar amounts in excess of the iron, where the degree of excess can result in free chitosan ligands that are not chelated to iron or any other metal that may be present. In another aspect, the degree of excess can depend on the number of chitosan ligands coordinated with the iron, where the ligands can be present in an amount to saturate the coordination potential of the iron, e.g., 2:1 chitosan to iron molar ratio. Alternatively, the chitosan ligand can be present only in an amount to chelate the iron, where the metal coordination potential is not saturated and retains the ability for further chelation. Accordingly, the iron and the chitosan ligand can be present at a chitosan to iron weight ratio from about 1:0.1 to about 1:0.001, and preferably from about 1:0.1 to about 1:0.01. In still another aspect, the chitosan to iron weight ratio can be from about 1:0.05 to about 1:0.02.

When selecting additional ingredients to be included in a composition containing an iron chitosan chelate, potential changes in the characteristics of the composition as a whole, as well as the potential physiological effects that can result, can be considered. Ascorbic acid, also known as Vitamin C, is water-soluble and important to many different physiological processes. For example, ascorbic acid can be important in the formation of collagen, which is a protein that gives structure to bones, cartilage, muscle, and blood vessels. Additionally, ascorbic acid also can help maintain capillaries, bones, and teeth. Further, not only can ascorbic acid aid in the absorption of iron, but it has also been implemented for use in the prevention of oxidation of unsaturated fatty acids and sex hormones. Accordingly, in one aspect of the present invention, ascorbic acid can be delivered with the iron chitosan chelate. The concentration present for daily dosage has been previously described. Depending in part on the weight of the dosage, amount of carrier to be delivered, in a solid dosage form, the ascorbic acid can be present in the composition at from about 1 wt % to about 10 wt %. The amount will typically be less by total weight percentage if in liquid dosage form, though the total amount of ascorbic acid delivered will be about the same.

Another ingredient that can optionally be delivered with the iron chitosan chelate, is zinc, typically in the form of a zinc source. Zinc is an essential mineral and is typically present in almost every cell because it can stimulate and enable the physiological activity of hundreds of enzymes. The presence of zinc can enable the body to promote and propagate important biochemical reactions. Zinc is especially useful for wound healing. Additionally, zinc can be beneficial for good health and normal growth during pregnancy, childhood, and adolescence, possibly because of its importance and role during DNA synthesis. Zinc supplementation can also enhance male factor sub-fertility, and conversely, zinc deficiency can cause growth retardation, hair loss, delayed sexual maturation, and/or impotence in males.

In accordance with this, the zinc source can be selected from the group consisting of zinc salts or complexes. Similarly, as with the iron salts, it is within the scope of the present invention that the zinc source be any zinc salt that can facilitate bioavailability of the zinc. In one aspect, and the zinc salt can be selected from the group consisting of zinc sulfate, zinc carbonate, zinc phosphonate, zinc acetate, and combinations thereof. Alternatively, zinc chelates can also be used, as are commercially available and are known in the art. The concentration that can be included for daily dosage has been previously described. Depending in part on the weight of the dosage to be delivered, in a solid dosage form, the zinc content can be present in the composition at from about 0.1 wt % to about 5 wt %. Again, the amount will typically be less by total weight percentage if in liquid dosage form due to the presence of the liquid carrier, though the total amount of ascorbic acid delivered will be about the same.

As kelp can aid in increasing Leydig cell proliferation, kelp can also be delivered with an iron source to improve testosterone stimulation. If included, depending in part on the weight of the dosage to be delivered, the kelp content can be present in the composition at from about 0.5 wt % to about 10 wt %.

To provide a specific example of a preferred formulation that can be prepared in accordance with embodiments of the present invention, a composition can be formulated in accordance with the present invention that includes a variety of formulation additives. For example, such a composition can be formulated in liquid form at from about 0.5 wt % to about 10 wt % of an organic acid, from about 1 wt % to about 10 wt % of chitosan, from about 0.05 wt % to about 1 wt % of iron, from about 1 wt % to about 10 wt % of ascorbic acid, from about 0.5 wt % to about 10 wt % of kelp, from about 0.1 wt % to about 5 wt % of zinc, and the balance can be other known fillers, carriers, liquids such as water, or other known inert ingredients. These ranges are exemplary preparatory ranges which can be used in accordance with an alternative embodiment of the present invention. When dried to solid form, the weight percentages can change, as is known in the art. If a liquid dosage form, e.g., solution or suspension, the composition can have a pH of about 4.0 to about 6.0, and can be administered as a daily dose of from about 4 mL to about 10 mL. Additionally, if in a solid form, e.g., granules, frozen powder, or spray-dried powder, equivalent solid dosages can be administered daily.

If delivered orally, a variety of oral dosage forms are well known to those of ordinary skill in the art, and specific formulation ingredients may be selected in order to provide a specific result. Examples of oral dosage forms include, without limitation, tablets, capsules, gel capsules, liquids, syrups, elixirs, and suspensions. Additionally, oral dosage forms encompass food preparations, such as fortified foods and beverages. If delivered by a means other than orally, other routes of administration can include transdermal or parenteral. A number of specific transdermal and parenteral dosage forms are well known to those of ordinary skill in the art. Examples of transdermal dosage forms include without limitation lotions, gels, creams, pastes, ointments, transmucosal tablets, adhesive devices, adhesive matrix-type transdermal patches, liquid reservoir transdermal patches, etc. Also, parenteral dosage forms can be formulated to provide efficient delivery of the composition to a subject, as would be known by one skilled in the art.

In another aspect, the compositions of the present invention can be delivered in a manner that is tailored for specific dosage regimens. The specific dosage form and amount of iron chitosan chelate, and other ingredients delivered can be designed to attain a desired concentration of any specific ingredient in a subject. Thus, those of ordinary skill in the art will be able to determine proper dosage amounts for specific individuals through routine monitoring and adjustment of the physiological effects of a given dosage. However, as a general matter, the compositions of the present invention can be administered in an amount and frequency sufficient to provide increased systemic testosterone levels in a subject compared to pre-administration testosterone levels of that subject. For example, the compositions of the present invention can be administered in an amount and at a frequency sufficient to treat and/or prevent testosterone deficiency, and/or treat/prevent any of the related signs and symptoms of testosterone deficiency, such as diminished libido, erectile dysfunction, muscle weakness, loss of body hair, depression, and other mood disorders.

### EXAMPLES

The following examples illustrate embodiments of the invention. However, it is to be understood that the following is only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following example provides further detail in connection with what is presently deemed to be a practical embodiment of the invention.

### Example 1

### Preparation of Iron Chitosan Chelate

An iron chitosan chelate is prepared by placing 50 grams of chitosan having a weight average molecular weight of 250,000 Mw with a 92% degree of acetylation into a reaction vessel containing 900 mL of a 1.75 vol % aqueous acetic acid solution with a temperature of 50° C., and dissolving the chitosan. To the solution containing dissolved chitosan, 100 mL of a chitosanase enzyme (35,000 CU/g) solution of concentration 150 mg/L is added to hydrolyze the chitosan. The hydrolysis process is mixed at a temperature 50° C. for 5 hours. The hydrolysis of chitosan is quenched by increasing the solution temperature to 80° C., which is maintained for 30 min. The solution is then cooled to room temperature by circulating cold water around the vessel. The solution is filtered with ultrafiltration to obtain the desired molecular weight range of low molecular weight chitosan. To the permeate containing the low molecular weight chitosan, 6.1 g of a ferrous lactate is added under agitation to obtain the iron chitosan chelate in a liquid solution form.

### Example 2

### Preparation of Iron Chitosan Chelate-Containing Composition

An iron chitosan chelate-containing composition is prepared by admixing the following ingredients: 1 L of the iron chitosan chelate solution of Example 1, 2.5 grams of ascorbic acid, 2.5 grams of kelp, and 0.5 grams of zinc acetate. Optionally, the composition is spray dried or freeze-dried to form a solid dosage form.

### Example 3

### Testosterone Stimulation

The effects of an iron chitosan chelate, chitosan, and iron on systemic testosterone levels in test groups of 8-week old Sprague Dawley rats is observed and compared to a control group. Each of the groups includes 8 rats. The rats of each group are orally administered a liquid composition twice a day, where group 1 receives a carrier composition in the form of a placebo, group 2 receives the carrier enhanced with 200 μL of 0.12 wt % iron lactate, group 3 receives the carrier enhanced with 200 μL of 1 wt % chitosan having a weight average molecular weight of 19,500 Mw, and group 4 receives the carrier enhanced with 200 μL of 1 wt % iron chitosan chelate (chitosan weight average molecular weight 19,500 Mw). The effects of the iron, chitosan, and iron chitosan chelate in systemic testosterone levels are compared to the systemic testosterone levels of the rats administered with the carrier placebo alone. After 7 days of dosing the 4 rat groups twice per day, the impact of the test groups compared to the control group on the rats testosterone levels is recorded, and is set forth in Table 1.

TABLE 1
Systemic Testosterone Levels

| | | % Increase |
|---|---|---|
| Dosage Type | Testosterone Level | from Placebo |
| Placebo | 370.6 ± 32.6 (pg/100 μL) | — |
| Iron | 358.1 ± 23.6 (pg/100 μL) | ˜0% |
| Chitosan | 467.5 ± 85.5 (pg/100 μL) | ˜26% |
| Iron Chitosan Chelate | 545.3 ± 88.6 (pg/100 μL) | ˜47% |

The results indicate that dosing rats for 7 days with 200 μL of 1 wt % iron chitosan chelate increases the testosterone levels by about 47% compared to the level of testosterone in the placebo group to a blood concentration of about 545 (pg/100 μL). Comparatively, the rats dosed with chitosan added to the carrier only increases the testosterone levels by about 26% to blood concentration levels of about 467 (pg/100 μL). Accordingly, the iron chitosan chelate and chitosan significantly increases the rat systemic testosterone levels compared to the control group and iron alone, but the iron chitosan chelate provided the greatest increase in testosterone levels.

### Example 4

### Iron Chitosan Chelate Dose Dependant Testosterone Stimulation

The effect of increasing the dose of iron chitosan chelate on systemic testosterone levels in 8-week old Sprague Dawley rats is observed. Specifically, 6 different groups of rats are administered a liquid dosage form iron chitosan chelates in increasing concentrations of 0.1 wt %, 0.5 wt %, 1 wt %, 2 wt %, 5 wt, and 10 wt % (chitosan weight average molecular weight 19,500 Mw). One group is administered a placebo. The effects of varying the iron chitosan chelate concentration for the orally administered 200 μL dosage twice a day on the change in systemic testosterone levels are compared to a control group of rats that are administered with only a placebo. After 7 days of dosing the seven rat groups 2 times per day, the impact of various amounts of the iron chitosan chelate on testosterone levels is recorded, and is set forth in Table 2.

TABLE 2
Iron Chitosan Chelate Dosages Relating To Testosterone Levels

| Iron Chitosan Chelate | | |
|---|---|---|
| Concentration in Liquid | | % Increase |
| Administered to Rats | Testosterone Levels | from Placebo |
| Placebo (0 wt %) | 291 ± 46 (pg/100 μL) | — |
| 0.1 wt % | 378 ± 73 (pg/100 μL) | ˜29.8% |
| 0.5 wt % | 385 ± 51 (pg/100 μL) | ˜32.3% |
| 1 wt % | 433 ± 71 (pg/100 μL) | ˜48.8% |
| 2 wt % | 419 ± 55 (pg/100 μL) | ˜43.6% |
| 5 wt % | 415 ± 54 (pg/100 μL) | ˜42.6% |
| 10 wt % | 403 ± 61 (pg/100 μL) | ˜38.4% |

The results indicate that dosing rats for 7 days with a 200 μL dosage twice a day with a 0.1 wt % iron chitosan chelate increased the average systemic testosterone level by about 29.8% compared to the testosterone level in the placebo group to a blood concentration of about 378 (pg/100 μL). Additionally, the rats dosed with the 0.5 wt % dosage showed about a 32.3% increase in systemic testosterone levels to a blood concentration of about 385 (pg/100 μL). Further, the rats dosed with the 1 wt % dosage increased systemic testosterone levels by about 48.8% in comparison with the placebo to a blood concentration of about 433 (pg/μL). The rats dosed with the 2 wt %, 5 wt %, and 10 wt % dosages increased the systemic testosterone levels by about 43.6%, 42.6%, and 38.4%, respectively, in comparison with the placebo to blood concentrations of about 418 (pg/100 μL), 415 (pg/100 μL), and 403 (pg/100 μL), respectively. Accordingly, increasing the concentration of iron chitosan chelates up to 1 wt % increased the level of systemic testosterone, where further increases in the iron chitosan chelate concentration did not substantially change the level of testosterone.

### Example 5

### Effect of Iron Concentration on Testosterone Stimulation

The effect of increasing the concentration of iron on systemic testosterone levels in 8-week old Sprague Dawley rats during the administration of iron chitosan chelates is observed. Specifically, five different groups of rats are administered iron chitosan chelates, where the chitosan concentration is fixed at 5 wt %, and where the iron in increasing concentrations of 0.05 wt %, 0.10 wt %, 0.15 wt %, 0.20%, and 0.25 wt % (chitosan weight average molecular weight 19,500 Mw, iron base). One group is administered a placebo. The effect of varying the iron concentration for the orally administered dosage on the change in systemic testosterone levels are compared to rats administered with only a placebo. After 7 days of dosing the seven rat groups with 200 μL twice a day, the impact of various concentrations of iron on testosterone levels is recorded, and is set forth in Table 3.

TABLE 3
Effect of Iron Concentration on Testosterone Levels

| | | % Increase from |
|---|---|---|
| Concentration of Iron | Testosterone Levels | Placebo |
| Placebo (0 wt %) | 253 ± 31 (pg/100 μL) | — |
| 0.05 wt % | 363 ± 44 (pg/100 μL) | ˜43% |
| 0.10 wt % | 401 ± 31 (pg/100 μL) | ˜58% |
| 0.15 wt % | 441 ± 35 (pg/100 μL) | ˜74% |
| 0.20 wt % | 434 ± 37 (pg/100 μL) | ˜71% |
| 0.25 st % | 439 ± 40 (pg/100 μL) | ˜74% |

The results indicate that dosing rats for 7 days with 200 μL twice a day of the 0.05 wt % iron dosage showed about a 43% increase compared to the placebo group to systemic testosterone levels of about 363 (pg/100 μL). Additionally, the rats dosed with the 0.10 wt % iron dosage increased systemic testosterone levels by about 58% in comparison with the placebo to a blood concentration of about 401 (pg/100 μL). The rats dosed with the 0.15 wt %, 0.20 wt %, and 0.25 wt % iron dosages increased the systemic testosterone levels by about 74%, 71%, and 74%, respectively, in comparison with the placebo to blood concentrations of 441 (pg/100 μL), 434 (pg/100 μL), and 439 (pg/100 μL), respectively. Accordingly, increasing the concentration of iron up to 0.15 wt % increased the level of systemic testosterone, where further increases in the iron concentration did not substantially change the level of testosterone.

It is to be understood that the above-described examples are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and/or preferred embodiments of the invention, it will be apparent to those of ordinary skill in the art that these examples not intended to be limiting in nature.

1. A method of increasing testosterone levels in a subject, comprising the steps of:
a) identifying a subject having a lower than average blood testosterone level; and
b) administering a therapeutically effective amount of an iron chitosan chelate to the subject such that the blood testosterone level of the subject is increased.

2. A method as in claim 1, wherein the weight average molecular weight of chitosan is from about 0.5 kDa to about 50 kDa.

3. A method as in claim 2, wherein the weight average molecular weight of chitosan is from about 0.5 kDa to about 20 kDa.

4. A method as in claim 1, further comprising co-administering a therapeutically effective amount of at least one additional ingredient to a subject, said ingredient selected from the group consisting of ascorbic acid, zinc, and kelp.

5. A method as in claim 4, wherein the ingredient is ascorbic acid.

6. A method as in claim 4, wherein the ingredient is kelp

7. A method as in claim 4, wherein the ingredient is zinc.

8. A method as in claim 7, wherein the zinc is a zinc salt.

9. A method as in claim 1, wherein the subject is a human.

10. A method as in claim 1, wherein the iron chitosan chelate is administered as a composition in an oral dosage form.

11. A method as in claim 10, wherein the oral dosage form is a liquid dosage form.

12. A method as in claim 10, wherein the oral dosage form is a solid dosage form.

13. A method of increasing testosterone levels in a subject, comprising the steps of:
a) identifying a subject having a lower than average blood testosterone level;
b) administering a therapeutically effective amount of chitosan to the subject; and
c) co-administering at least one ingredient selected from the group consisting of ascorbic acid, iron, zinc, and kelp to the subject such that the blood testosterone level of the subject is increased.

14. A method as in claim 13, wherein the weight average molecular weight of chitosan is from about 0.5 kDa to about 50 kDa.

15. A method as in claim 14, wherein the weight average molecular weight of chitosan is from about 0.5 kDa to about 20 kDa.

16. A method as in claim 13, wherein the at least one ingredient is ascorbic acid.

17. A method as in claim 13, wherein the at least one ingredient is kelp.

18. A method as in claim 13, wherein the at least one ingredient is iron.

19. A method as in claim 18, wherein the iron is an iron salt.

20. A method as in claim 13, wherein that at least one ingredient is zinc.

21. A method as in claim 20, wherein the zinc is a zinc salt.

22. A method as in claim 13, wherein the subject is a human.

23. A method as in claim 13, wherein the chitosan is administered as a composition in an oral dosage form.

24. A method as in claim 23, wherein the oral dosage form is a liquid dosage form.

25. A method as in claim 23, wherein the oral dosage form is a solid dosage form.

26. A method as in claim 13, wherein the chitosan is administered in the form of a chelate.

27. A method as in claim 26, wherein the chelate is an iron chitosan chelate.

28. A composition for enhancing testosterone stimulation, comprising:
an iron chitosan chelate, wherein the chitosan has a weight average molecular weight from about 0.5 kDa to about 50 kDa; and
at least one ingredient selected from the group consisting of ascorbic acid, kelp, and zinc.

29. A composition as in claim 28, wherein the chitosan has a weight average molecular weight from about 0.5 kDa to about 20 kDa.

30. A composition as in claim 28, wherein the weight ratio of the total amount of chitosan to the total amount of iron is from about 1:0.001 to about 1:0.1.

31. A composition as in claim 30, wherein the weight ratio of the total amount of chitosan to the total amount of iron is from about 1:0.05 to about 1:0.02.

32. A composition as in claim 28, further comprising an organic acid selected from the group consisting of acetic acid, citric acid, malic acid, lactic acid, and combinations thereof.

33. A composition as in claim 28, wherein the composition is in a solid dosage form.

34. A composition as in claim 33, wherein the solid dosage form is selected from the group consisting of granules, frozen powder, and spray dried powder.

35. A composition as in claim 33, wherein the weight ratio of the total amount of chitosan to the total amount of ascorbic acid is from about 1:0.01 to about 1:0.1.

36. A composition as in claim 33, wherein the weight ratio of the total amount of chitosan to the total amount of kelp is from about 1:0.005 to about 1:0.1.

37. A composition as in claim 33, wherein the weight ratio of the total amount of chitosan to the total amount of zinc is from about 1:0.001 to about 1:0.05.

38. A composition as in claim 33, wherein the chitosan is present in the solid dosage form at from 40 wt % to about 80 wt %, and the iron is present in the solid dosage form at from 0.1 wt % to about 4 wt %.

39. A composition as in claim 28, wherein the composition is in a liquid dosage form, and the pH of the liquid dosage form is from about 4.0 to about 6.0.

40. A method of making an iron chitosan chelate, comprising:
hydrolyzing a chitosan source to form a hydrolyzed chitosan mixture, said chitosan mixture including a desired portion and a remainder portion;
substantially separating the desired portion from the remainder portion; and
chelating the desired portion with iron.

41. A method as in claim 40, wherein the hydrolyzing step occurs by mixing the chitosan source with chitosanase in the presence of an organic acid.

42. A method as in claim 41, wherein the organic acid is acetic acid.

43. A method as in claim 40, wherein the hydrolyzing step occurs by mixing the chitosan source with chitosanase in the presence of hydrochloric acid.

44. A method as in claim 40, further comprising the step of heating the mixture prior to the step of separating to stop hydrolysis of the chitosan.

45. A method as in claim 40, wherein the iron is provided by an iron salt.

46. A method as in claim 45, wherein the iron salt is selected from the group consisting of ferrous sulfate, ferrous lactate, and combinations thereof.

47. A method as in claim 40, wherein the step of separating is performed by ultrafiltration.

48. A method as in claim 40, wherein the desired portion of the chitosan mixture includes chitosan ligands having a weight average molecular weight from about 0.5 kDa to about 50 kDa.

49. A method as in claim 48, wherein the desired portion of the chitosan mixture includes chitosan ligands having a weight average molecular weight from about 0.5 kDa to about 20 kDa.

50. A method as in claim 40, said method carried out in solution to form a liquid.

51. A method as in claim 40, further comprising the step of drying the solution to form solid particulates.

52. A method of preparing a composition for enhancing testosterone stimulation, comprising:
preparing an iron chitosan chelate as in claim 40; and
combining the iron chitosan chelate with at least one ingredient selected from the group consisting of ascorbic acid, kelp, and zinc.

53. A method as in claim 52, further comprising the step of formulating the composition as a solid dosage form.

54. A method as in claim 52, further comprising the step of formulating the composition as a liquid dosage form.
